(19) United States
(12) Patent Application Publication
(10) Pub. No.: US 2013/0310650 A1
(43) Pub. Date: Nov. 21, 2013

(54) LARYNGOSCOPY

(71) Applicant: Paul Christopher Hales, (US)

(72) Inventors: Paul Christopher Hales, Mount Eliza (AU); Michael Chijoff, Footscray (AU)

(73) Assignee: Paul Christopher Hales, Mount Eliza (AU)

(21) Appl. No.: 13/693,957

(22) Filed: Dec. 4, 2012

Related U.S. Application Data
(63) Continuation of application No. PCT/AU2011/000699, filed on Jun. 3, 2011.

(30) Foreign Application Priority Data
Jun. 4, 2010 (AU) 2010902464

(51) Int. Cl. A61B 1/267; A61M 16/04; A61B 1/00; A61B 1/06; A61B 1/04

(52) U.S. Cl. CPC A61B 1/267; A61B 1/06; A61B 1/04; A61B 1/00165; A61M 16/0488
USPC 600/188; 600/196; 600/194

(57) ABSTRACT

There is disclosed a laryngoscope comprising a blade having an adjustable curvature and means for adjusting the curvature which is operable during insertion of the blade into the airway of a patient such that the blade lifts the tongue and anterior structures to facilitate the insertion. There is also disclosed a laryngoscope fitting which defines a guide along which an endotracheal tube may be fed to intubate a patient following insertion of the laryngoscope into the patient's airway, the fitting being releasable from the blade to permit withdrawal of the blade from the fitting and thus from the airway.

### FIELD OF THE INVENTION

The present invention relates generally to improvements in laryngoscopy, and more particularly to a laryngoscope, a fitting for a laryngoscope, for facilitating endotracheal intubation, and methods of laryngoscopy.

The invention has application to both human and animal patients.

### BACKGROUND

A laryngoscope is a medical instrument used to obtain a view of a patient's vocal cords and glottis, usually in order to allow insertion of a tube in the patient's trachea (“endotracheal intubation”) for the purposes of providing a patent airway to facilitate gas exchange. This most commonly occurs during general anaesthesia and in the intensive care setting.

Direct laryngoscopy, in which a laryngoscope is used to obtain a direct view of the vocal cords, is often carried out using a straight laryngoscope blade, known as a “Miller” blade, or a curved blade, known as a “Macintosh” blade. A laryngoscope L having a Macintosh blade B is illustrated in FIG. 1, the blade B being formed with an anterior blade portion A and a posterior flange F arranged at right angles to the portion A. The laryngoscope further comprises a handle H and a light S mounted to the flange F.

Direct laryngoscopy using a Macintosh blade typically comprises the following steps:
1. flexion of the lower cervical vertebrae of the neck and extension of the head at the atlanto-occipital joint to align the oral, pharyngeal and laryngeal axes;
2. opening of the mouth with the right hand (laryngoscopes being designed for left-handed use) and insertion of the blade into the right side of the mouth;
3. displacement of the tongue and anterior structures upwards and to the left using the flange F as the blade B runs along the contour of the tongue; and
4. insertion of the tip of blade B into the vallecula (which is the space between the tongue and the epiglottis).

Direct laryngoscopy using alternative types of blade is carried out in a generally similar manner though in the case of a Miller blade, the blade tip is inserted posterior to the epiglottis, and in the case of a McCoy blade, the epiglottis may be displaced anteriorly by the tip of the blade.

Endotracheal intubation, by insertion of the endotracheal tube through the thus exposed vocal cords, may then be carried out.

A number of factors can make conventional direct laryngoscopy and intubation difficult. Approximately one in 65 intubations is difficult. The incidence of failed intubation is estimated at 1:300, rising to 1:30 in the obstetric population. Sequelae include trauma to the airway and teeth, aspiration of gastric content, hypoxaemia (low oxygen levels in the blood), potentially leading to death or permanent organ damage. In 2005, Davidson et al. estimated there were 21 million general anaesthetics administered to adults and children in the US alone. Factors which cause difficulty in laryngoscopy and intubation are classified as those relating to the anaesthetist/anaesthetic equipment or patient factors. Inadequate preparation, inexperience and poor technique are common causes of difficult intubation. Equipment malfunction, lack of availability of aids to intubation or trained assistance may lead to serious complications. Patient factors are classified as congenital or acquired. Congenital syndromes (such as Down's, Pierre-Robin, Treacher-Collins and Marfan's) are associated with difficult intubation.

Acquired factors include the following.

1. Reduced Neck Movement
This occurs in trauma (including application of a hard collar), cervical fusion (in the case of which the vertebrae are fixed), and in conditions such as rheumatoid and osteo-arthritis and ankylosing spondylitis. Movement of an unstable neck can lead to permanent neurological injury.

2. Reduced Mouth Opening
This occurs in trismus (muscle spasm resulting in inability to mouth open) due to infection, fracture or tetanus. Trauma, abscesses, radiotherapy, surgery, rheumatoid arthritis, and ankylosing spondylitis all reduce jaw movement and mouth opening.

3. Airway Problems
(i) Pathological
Oedema (soft-tissue swelling) in infection, anaphylaxis (severe allergic reactions), trauma or burns.
Obstruction of the airway by trauma and subsequent haemorrhage, foreign body, tumours or polyps.
Compression of the airway by goitre (large thyroid gland) or bleeding.
Narrowing of the airway due to scarring post-radiotherapy, surgery or burns.
(ii) Anatomical
Short, muscular neck and reduced atlanto-occipital distance make neck extension difficult or impossible.
Protruding teeth (especially incisors), a receding lower jaw, poor mobility or increased anterior depth of the mandible and an anterior larynx are all associated with difficult laryngoscopy and intubation.

In the event of a failed intubation, if oxygenation of the patient is not possible via other means (bag and mask ventilation, laryngeal mask airway etc.) a cricothyrotomy or tracheostomy must be performed. In an emergency situation this procedure carries a very high mortality.

The term “patient” as used herein, unless context requires otherwise, is broad enough in scope as to refer to a human patient or an animal patient.

### SUMMARY OF THE INVENTION

According to a first aspect of the present invention, there is provided a laryngoscope comprising a blade having an adjustable curvature and means for adjusting the curvature which is operable during insertion of the blade into the airway of a patient such that the blade lifts the tongue and anterior structures to facilitate the insertion.

A direct view of the patient's vocal cords can thus be readily established.

In the preferred embodiments of the invention, the curvature adjustment means comprises a curvature adjustment mechanism, which is preferably operable to effect axial compression along an anterior side of the blade to increase blade curvature but alternatively may be operable to effect axial tension along a posterior side of the blade to increase blade curvature.

Advantageously, adjusting the curvature of the blade to lift the tongue and anterior structures can substantially eliminate the need for the operator of the laryngoscope to pull on the laryngoscope handle axially to draw the entire laryngoscope anteriorly, and thus reduce or eliminate forces on the cervical vertebra, which are often unavoidable in direct laryngoscopy and which can worsen neck injuries and cause paralysis.

Preferably, the blade comprises a proximal section adjacent the handle and a distal section extending from the proximal section, the distal section being angularly displaceable relative to the proximal section via the means for adjusting curvature whereby the blade curvature is altered. Preferably, the distal section has a length which is approximately 50% of the length of the blade. In a preferred embodiment of the invention, the distal section has a length which is approximately 45% to 55% of the blade length. In one embodiment, the distal section length is approximately half of the blade length.

Preferably, the proximal section is fixed with respect to the handle.

Preferably, the curvature of the distal section is adjustable to adjust the curvature of the blade.

Preferably, the blade includes a plurality of portions therealong which are displaceable by the adjustment means at rates which increase progressively from a proximal one of the portions to a distal one of the portions. Accordingly, a tip of the blade can be the part of the blade which is displaced through the greatest angle for a given degree of positive (curvature-increasing) adjustment via the adjustment means. Advantageously, the tip can, by operation of the adjustment means, be appropriately orientated, during insertion of the blade into the buccal cavity/pharynx, to point towards the larynx, being able in so doing to displace the tongue/anterior structures, to be received under the epiglottis, greatly facilitating the laryngoscopy.

Preferably, the means for adjusting the curvature includes an actuating means which is arranged to be operated with the same hand as that which is holding the laryngoscope handle. In a preferred embodiment of the invention, the actuating means comprises an actuator which is operable by a digit of the hand whilst the handle is grasped by the hand. The actuator may comprise, for example, a button, slider or switch. Preferably, the handle is configured to be grasped such that the operator's fingers extend around a front portion thereof and the operator's thumb is disposed against a rear portion thereof, and the actuator is disposed at or adjacent the rear portion to be operable by the thumb. In one embodiment of the invention, the actuator is operable by being translationally displaced along a longitudinal axis of the handle. In another embodiment of the invention, the actuator is rotary e.g. comprising a roller, wheel or dial, which is preferably rotatable about an axis extending transverse, and preferably perpendicular, to longitudinal axes of both the blade and handle. Preferably, the rotary actuator is thumb-operable.

In one embodiment of the invention, the mechanism is unpowered. In another embodiment of the invention, the mechanism may be powered; for example, the mechanism may comprise at least one electrically powered motor, such as a servo-motor, operable via the actuator, to effect relative angular displacement between portions of the blade and thus the adjustment of blade curvature.

Preferably, the adjustment mechanism is configured to be locked after each operation thereof to retain blade curvature. In one embodiment of the invention, in which the adjustment mechanism is unpowered, that mechanism is, to this end, provided with a ratchet. In another embodiment of the invention, in which the adjustment mechanism may be similarly unpowered, the actuator is configured to be urged against a resilient bias in a first direction, which may be rotational or translational, to unlock the mechanism and movable in a second direction, which may be rotational or translational, to effect curvature adjustment.

In one embodiment of the invention, the actuator is operable to adjust curvature along a continuum. In another embodiment of the invention, the actuator is operable to adjust the curvature incrementally.

According to a preferred embodiment of the invention, the blade comprises a plurality of segments arranged in end-to-end relation in a direction from the proximal end of the blade to the distal end of the blade, and at least one joint, the or each joint interconnecting the segments in the or each respective pair of adjacent segments, a distal segment in the or each pair being engaged with the means for adjusting curvature, whereby operation of the means for adjusting curvature effects articulation of the distal segment(s) about the joint(s). Preferably, the blade comprises at least three segments and thus at least two joints. The blade profile can then approximate a curve more closely than if the blade comprised only two segments and a single joint. In a preferred embodiment of the invention, the blade comprises four segments and thus three joints. It is envisaged that this number of segments and joints offers the optimum compromise between simplicity of construction and both smoothness of curvature and fineness of curvature adjustment.

Preferably, the segments are separately formed. Alternatively, the blade may be of unitary/single-piece construction and resiliently flexible, so that the segments are integrally formed (and may thus be notional segments) and the joints are defined by portions of the blade (and thus may be notional joints) which resiliently flex/deform upon operation of the means of for adjusting curvature to increase the curvature, the resilient flexibility of the blade, in such an embodiment, preferably then biasing the blade into a relaxed configuration, which is preferably a straight configuration.

In a preferred embodiment of the invention, the blade is configured to lock whereby increasing of its curvature beyond a maximum degree is precluded. Preferably, to this end, the segments are configured to lock. Preferably, adjacent ends of the segments in the or each said pair are formed with faces which are arranged to abut upon said maximum degree being reached.

In one preferred embodiment of the invention, the ratios of the lengths of the four segments in the direction from the proximal end to the distal end of the blade are approximately 16:6:4:3; for example, the lengths of the segments in the direction from the proximal end to the distal end may be approximately 8 cm, 3 cm, 2 cm and 1.5 cm. In another preferred embodiment of the invention, the lengths of the four segments the direction from the proximal end to the distal end of the blade are approximately 6.0:3.0:2.3:1.6; for example, the lengths of the segments in the direction from the proximal end to the distal end may be approximately 6.0 cm, 3.0 cm, 2.3 cm and 1.6 cm.

In a preferred embodiment of the invention, the or each joint is biased such that the blade reverts to a relaxed configuration as the curvature adjustment means is returned to a minimum position. Preferably, the relaxed configuration is a straight configuration.

In a preferred embodiment of the invention, the means for adjusting the blade curvature comprises a mechanism including at least one tendon which is anchored at a distal end thereof to the distal segment in a respective said pair of adjacent segments and extending across, and anterior of, a respective articulation axis of the distal segment defined by the respective joint, and which is coupled at a proximal end thereof to an actuating means of the laryngoscope which is operable to effect tensioning of the tendon(s) and thus articulation of the distal segment(s) about the joint(s). The or each tendon may be either directly or indirectly connected to the actuating means. The or each tendon may comprise a cable.

In a preferred embodiment of the invention, the tendons extend posterior of the blade anterior surface. Preferably, in this embodiment, the tendons extend within the blade.

In one embodiment of the invention, said tendons extend along the blade/segments in layered relation. In another embodiment of the invention, said tendons extend along the blade/segments in side-by-side relation.

Preferably, the tendon or tendons anchored to the or each distal segment is or are symmetrically disposed with respect to a central axis of the blade or a central plane passing through the blade orthogonal thereto. In one embodiment of the invention, the or each tendon extends along the central axis of the blade.

In one embodiment of the invention, the or each tendon extends internally in the blade. In another embodiment of the invention, the or each tendon extends exteriorly of the blade or comprises sections which extend exteriorly of the blade.

In the preferred embodiments of the invention, the laryngoscope is configured with retaining means which retains the tendon(s) in close proximity to the segments along the length of the blade. Preferably, the retaining means comprises at least one retainer received over the tendon(s), with respect to which the tendon/s is/are axially movable.

Preferably, the retaining means comprises at least one retainer on the proximal segment in the or each said pair received over the tendon(s) anchored to the distal segment in that pair, with respect to which retainer(s) the tendon(s) are axially movable, preferably slidably movable. In one embodiment of the invention, the retainer(s) on the or each proximal segment effect/s anchorage the tendon(s) anchored to that segment.

Preferably, the or each retainer is configured to permit limited anterior displacement of the tendon(s) over which it is received and which is/are axially movable with respect thereto, during drawing of the tendon(s) to increase the curvature of the blade.

In one embodiment of the invention, the or each retainer comprises a member attached to the respective segment. In another embodiment of the invention, the or each retainer is integrally formed with the respective segment. In the latter embodiment, the or each retainer is preferably defined by a passage through the respective segment, opposite ends of the passage opening out onto an anterior surface of the segment.

The blade may comprise a generally smooth, flexible or pliable exterior casing or covering which covers the segments and/or joints. Preferably, the casing/covering is resiliently flexible. In a preferred embodiment of the invention, the casing/covering covers the tendons.

Alternatively, the blade may be provided without such a casing/covering. In a preferred embodiment of the invention, the anterior surface of the blade is defined by anterior surfaces of the segments.

In a preferred embodiment of the invention, the adjustment mechanism comprises a pulley/roller/sheave system between the blade and the actuating means, over which the or each tendon is trained. The system may comprise one or more pulleys/rollers/sheaves, preferably disposed in the handle.

Preferably, the blade comprises at least three segments and the distal segments in the pairs of adjacent segments are displaceable by the adjustment means at rates which increase progressively from a proximal one of the distal segments to a distal one of the distal segments. Accordingly, a tip of the blade can be the part of the blade which is displaced through the greatest angle for a given degree of adjustment via the adjustment means. Advantageously, the tip can, by operation of the adjustment means, be appropriately orientated, during insertion of the blade into the airway, to point towards the larynx, being able in so doing to displace the tongue/anterior structures, to be received under the epiglottis, greatly facilitating laryngoscopy. Preferably, the adjustment mechanism comprises said tendons, and the tendons engage the segments at differential positions to enable the adjustment at rates which increase progressively from a proximal one of the distal segments to a distal one of the distal segments.

Advantageously, the plurality of tendons, resulting from there being at least three segments, can contribute to the reinforcement provided to the blade as it lifts the tongue and anterior structures (at which time the tendons are loaded as a result of the lifting action), and thus strength of the blade. Preferably, the laryngoscope comprises an actuating means which is operable to draw all of the tendons together, i.e. as one.

In a preferred embodiment of the invention, the curvature adjustment means/mechanism comprises a motor which is operable to draw the tendon(s). In this embodiment, the actuating means, which may, for example, comprise a switch, is operable to activate the motor. The curvature adjustment mechanism may further comprise a spool, which is driven by the motor, onto which the or each tendon is wound when the curvature is positively adjusted (i.e. adjusted in a curvature-increasing direction), the spool preferably being disposed or mounted in the handle.

In a preferred embodiment of the invention, the blade is provided with a resilient bias towards a relaxed condition/configuration, against which the curvature adjustment mechanism is operable to increase curvature, the bias being operative to reduce blade curvature upon unlocking of the adjustment mechanism and/or manual or powered driving of that mechanism in a reverse direction. The blade may be resiliently flexible to be afforded said bias.

In one preferred embodiment of the invention, the or each joint comprises a living hinge. The living hinge may be resiliently flexible to afford the blade said bias. The or each living hinge may be formed integrally with or separately from the adjacent segments.

The or each joint may comprise at least one ligament interconnecting the segments. Preferably, the or each joint is then defined by a pair of ligaments disposed adjacent opposite lateral sides of the blade.

The or each joint/ligament/living hinge may be defined by at least one resiliently flexible member to which the segments in the respective pair are fixed. Preferably, the or each resiliently flexible member comprises a spine member extending substantially the length of the blade to reinforce the blade.

In another preferred embodiment of the invention, the or each joint comprises a hinge via which the segments are pivotally interconnected.

Preferably, the blade is configured to support the tongue along substantially the entirety of the portion thereof which is angularly displaceable to effect the adjustment of blade curvature.

Preferably, the blade is configured to assume the profile of a respective substantially smooth arc in each of a plurality of adjustment positions of the adjustment means.

It will be appreciated that the profile along the length of the blade in the preferred embodiments is defined by a plurality of straight sections of blade arranged end-to-end and at different angles to approximate a curve, so that the term “curvature” is to be construed as a reference not only to a profile which is a substantially true curve but also one which generally approximates a true curve.

Preferably, the blade is long and thin (i.e. of small depth). Preferably, the blade thickness/depth does not exceed approximately half a centimetre/5 mm. The blade may have a uniform thickness along its length or be of progressively reducing thickness in the direction from its proximal end to its distal end.

In the preferred embodiments of the invention, the blade is configured in the form of an elongate spatula blade or elongate tongue. Preferably, the blade is generally planar.

Preferably, the blade is of progressively reducing width in a direction from a proximal end thereof to a distal end thereof.

Preferably, the blade is has a structure which is substantially symmetrical laterally about a longitudinal axis thereof.

Preferably, the laryngoscope is configured for both left-hand and right-hand operation. Preferably, the laryngoscope has, to this end, a structure which is substantially symmetrical bilaterally.

Preferably, the laryngoscope includes a light source operable to provide illumination to facilitate insertion of the blade.

Preferably, the laryngoscope includes a camera operable to provide a view of the airway, especially ahead of the blade, to facilitate insertion of the blade. Preferably, the camera comprises a fibre-optic wire extending along the blade and having an input end positioned at a distal end of the blade. In one embodiment of the invention, the camera has an output which is adapted to be coupled to a video screen remote from the laryngoscope. In another embodiment, the laryngoscope comprises a video screen coupled to the camera, to which screen the camera has an output. In the latter embodiment, the video screen may be provided on the handle, preferably on a posterior part of the handle.

In preferred embodiments of the invention, the laryngoscope is provided with a power source which powers the camera/light/screen.

According to a second aspect of the present invention, there is provided a laryngoscope provided with a fitting which in use is received by the blade of the laryngoscope to define a guide along which an endotracheal tube may be fed to intubate a patient following insertion of the laryngoscope blade into the patient's airway to provide a view of the patient's vocal cords, the fitting being releasable from the blade to permit withdrawal of the blade from the fitting and thus from the airway.

Preferably, the laryngoscope is that as described above, which accords with the first aspect of the invention.

According to a third aspect of the present invention, there is provided a fitting for a laryngoscope which when in use is received by the laryngoscope blade to define a guide along which an endotracheal tube may be fed to intubate a patient following insertion of the laryngoscope into the patient's airway to provide a view of the patient's vocal cords, the fitting being releasable from the blade to permit withdrawal of the blade from the fitting and thus from the airway.

Preferably, the fitting is received over the blade. Preferably, the fitting comprises a passage in which the blade is received (“the blade passage”). Preferably, the blade passage is open at a proximal end thereof to permit the blade to be inserted into and withdrawn from the blade passage.

Preferably, the blade passage is closed at a distal end thereof. Preferably, the fitting comprises a substantially transparent wall at the distal end of the blade passage to allow operation of a light and/or camera at a distal end of the blade to facilitate laryngoscopy.

Preferably, the blade passage is defined by a sleeve. Preferably, the fitting forms a liquid/pathogen-impermeable barrier over the blade.

Preferably, the fitting includes a passage which, when the fitting is in use, extends in alignment with the blade and is open at opposite ends (“the guide passage”), the guide passage being arranged for insertion of the tube therethrough. Preferably, the guide passage is defined by a sleeve.

Preferably, the fitting comprises a sheath.

Preferably, the guide passage is arranged to be disposed on a posterior side of the blade.

Preferably, the fitting is adapted to conform closely to the blade.

Preferably, the fitting is non-rigid. Preferably, the fitting is pliable or flaccid.

Preferably, the fitting is formed from an elastomer. In one embodiment of the invention, the fitting is formed from latex.

Preferably, the blade and fitting as received thereby define a blade structure having transverse (width/thickness) dimensions which are close to those of the blade. In particular, in the preferred embodiments of the invention, the fitting, as fitted to the blade, is configured with a small depth (along a thickness axis of the blade), so as not to hinder, to any appreciable extent, insertion of the blade.

In one embodiment of the invention, the fitting is formed from polyurethane.

Preferably, the fitting is securable to the tube to prevent axial movement of the fitting relative to the tube following withdrawal of the blade from the fitting. Preferably, the fitting is frictionally engageable with the tube to be secured thereto. In accordance with a preferred embodiment of the invention, string can be wound around a proximal end of the fitting to effect the frictional engagement.

Preferably, the fitting is releasably attachable to the laryngoscope. Preferably, the fitting is attachable to the laryngoscope adjacent a proximal end of the blade. Preferably, the fitting is adapted to be stretched in a proximal direction for attachment to the laryngoscope.

According to a fourth aspect of the present invention, there is provided a laryngoscopy procedure, comprising inserting the blade of a laryngoscope into the airway of a patient, and adjusting the curvature of the blade such that the blade lifts the tongue and anterior structures to facilitate the insertion.

Preferably, adjustment of the curvature of the blade is effected by the user's hand which is grasping the laryngoscope handle to insert the blade.

Preferably, the method comprises using a laryngoscope as defined above.

According to a fifth aspect of the present invention, there is provided an endotracheal intubation procedure comprising:
effecting receipt, by the blade of a laryngoscope, of a fitting to define a guide along the blade;
inserting the blade into the airway of a patient to obtain a view of the patient's vocal cords;
feeding the tube along the guide to intubate the patient; and
withdrawing the blade from the airway in a manner which releases it from the fitting, whereby the fitting remains inserted in the airway with the tube.

Preferably, the procedure comprises securing the fitting to the tube after withdrawal of the blade to preclude relative axial movement between the fitting and tube.

Preferably, insertion of the blade into the airway of the patient is effected via the procedure according to the fourth aspect of the invention.

### BRIEF DESCRIPTION OF THE DRAWINGS

The invention will be described, by way of non-limiting example only, with reference to the accompanying drawings, in which:

FIG. 1 is a perspective view of a conventional laryngoscope incorporating a Macintosh blade;

FIG. 2 is a side section view showing use of the laryngoscope of FIG. 1;

FIG. 3 is a perspective view of a laryngoscope according to a first preferred embodiment of the present invention;

FIG. 4 is a perspective view of a sheath-like fitting receivable over the blade of the laryngoscope of FIG. 3 to guide an endotracheal tube into a patient's larynx;

FIG. 5 is a view of a distal end of the fitting of FIG. 4;

FIG. 6A is a plan view of the blade of the laryngoscope of FIG. 3, in which internal details of the blade are shown in broken lines;

FIG. 6B shows configurations of retainers, tendons and segments within the blade at positions E-E, F-F, G-G and H-H marked in FIG. 6A;

FIG. 7 is a plan view of the sheath of FIG. 5;

FIG. 8 is a side cut-away view of the laryngoscope of FIG. 3;

FIG. 9 is a side/longitudinal cross-sectional view of the portion A marked in FIG. 8;

FIG. 10 is a transverse cross-sectional view of the blade of the laryngoscope at position I-I marked in FIG. 9;

FIG. 11 is a side view showing variation in the curvature of the blade of the laryngoscope of FIG. 3;

FIG. 12 is a perspective view showing fitting and attachment of the fitting of FIG. 4 to the laryngoscope of FIG. 3;

FIGS. 13 to 16 are schematic sectional views showing stages a laryngoscopy procedure using the laryngoscope of FIG. 3;

FIGS. 17 and 18 show an intubation procedure, which follows the procedure depicted in FIGS. 13 to 16;

FIG. 19 is a front perspective view showing the handle/actuator assembly and laid segment assembly of a laryngoscope according to a second? embodiment of the present invention;

FIG. 20 is a rear perspective view of the arrangement shown in FIG. 19;

FIG. 21 is a further front perspective view of the arrangement shown in FIG. 19, showing different degrees of blade curvature;

FIG. 22 is a perspective view corresponding to FIG. 19, additionally showing tendons of the laryngoscope;

FIG. 23 is a front view of the assembly of FIG. 19;

FIG. 24 is a view through section B-B marked in FIG. 23 though additionally showing one of the tendons and a fibre-optic cable of the laryngoscope;

FIG. 25 is a perspective view showing a resiliently flexible casing of the blade received over the segment assembly of the arrangement shown in FIG. 19; and

FIG. 26 is a front perspective view of a laryngoscope according to a third preferred embodiment of the present invention.

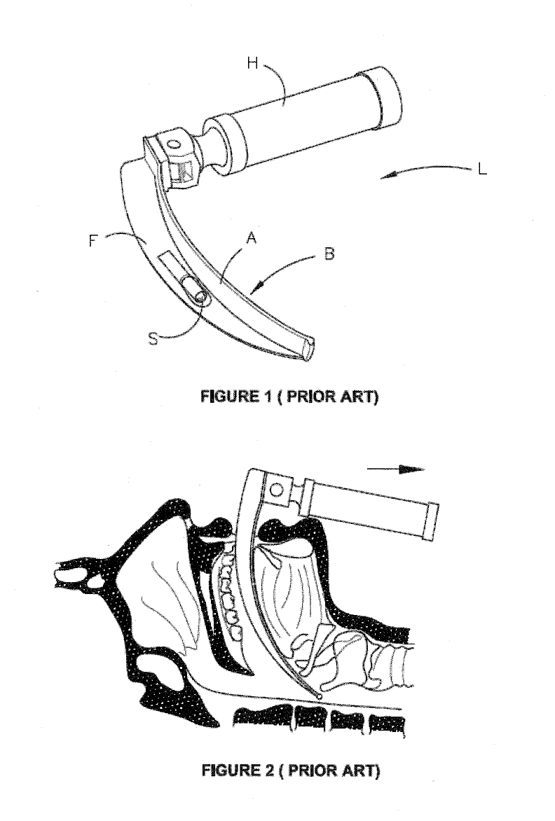

### DETAILED DESCRIPTION OF THE PREFERRED EMBODIMENTS

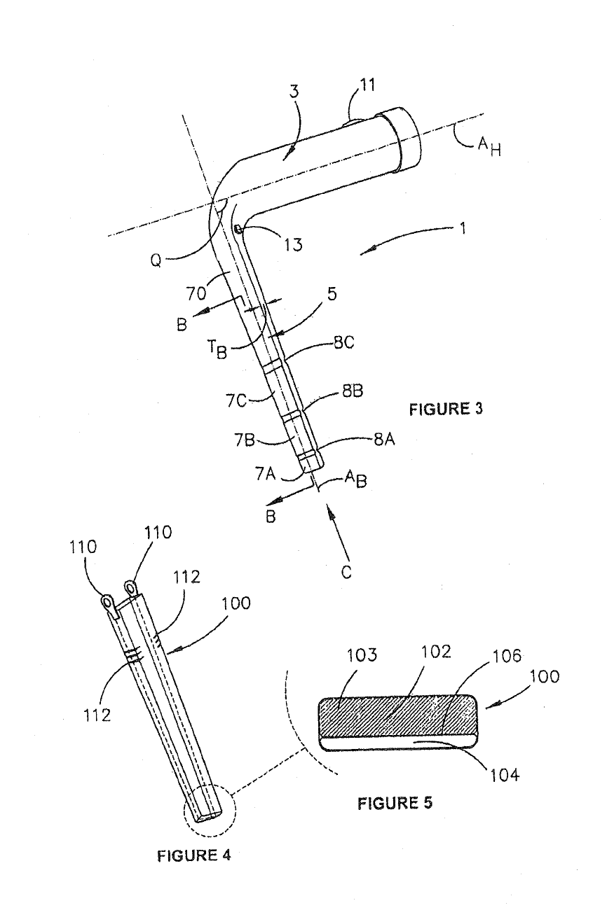

A laryngoscope 1 according to a first preferred embodiment of the present invention is shown in FIG. 3. The laryngoscope 1 comprises a generally cylindrical handle 3 and a blade 5 the exteriors of which are moulded from suitable plastic to form a unitary casing 9 which defines the entirety of the blade exterior. The blade 5 is configured in the manner of a tongue or in the form of an elongate spatula blade, and comprises, in the direction from the distal end to the proximal end thereof, four segments, 7A, 7B, 7C and 7D. The blade 5 further comprises three flexible joints, namely joint 8A, which interconnects segments 7A and 7B, joint 8B, which interconnects segments 7B and 7C, and joint 8C, which interconnects segments 7C and 7D. The joints 8A, 8B and 8C will be described in further detail later.

The laryngoscope 1 further comprises a mechanism for adjusting the curvature of the blade 5, which is operable by means of an actuator 11 which in this embodiment is located at a rear position on the handle 3 adjacent the free (upper) end thereof. The curvature adjustment mechanism, including actuator 11, will be described in further detail later.

Referring to FIGS. 3 and 4, provided on opposite sides of the laryngoscope 1 adjacent a proximal end of the blade 5 are respective lugs 13 for securement of a sheath 100 to the blade 5 to facilitate intubation, as will be described in further detail later. FIG. 3 shows the blade 5 in a straight, relaxed, configuration, in which it extends along longitudinal axis A_{B}, which axis forms an included angle with the longitudinal axis A_{H} of the handle 3 of approximately 120°, which will permit limited anterior translation of the laryngoscope 1 along the axis A_{H} to facilitate feeding of an endotracheal tube through the sheath 100, as will be described in further detail later.

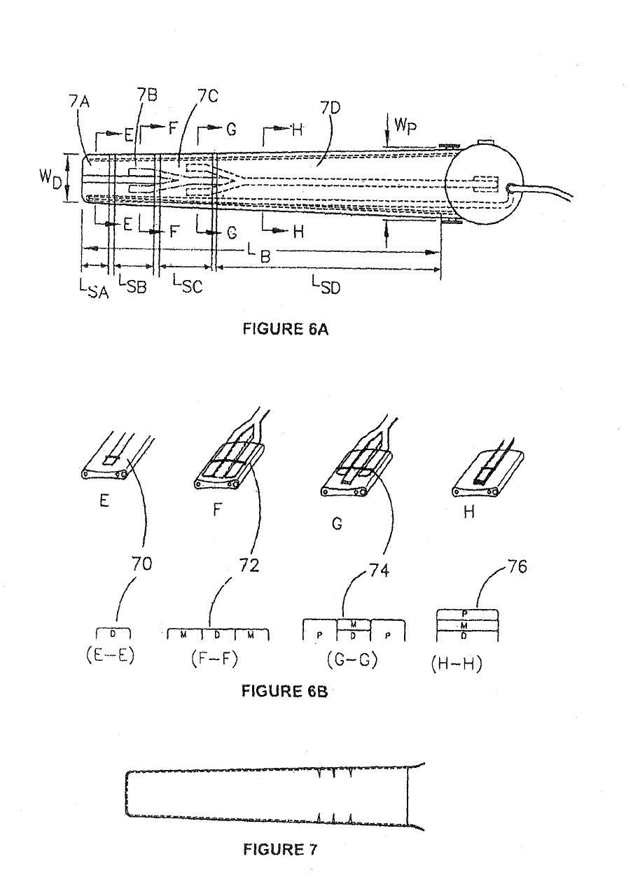

Each of the segments 7A to 7D has a flat, planar configuration and a depth/maximum thickness T_{B} of approximately 5 mm so that the blade has a substantially uniform thickness along its length. Referring to FIG. 6A, the blade has a structure which is substantially symmetrical about central longitudinal axis A_{B} and configured with a laterally inward taper in the direction from the proximal end to the distal end so as to be of progressively reducing width, the width W_{D} at the blade tip being approximately 2 cm to approximately 3 cm and the width W_{P} at the proximal blade end being approximately 3 cm to approximately 4 cm, whereby the blade 5 has sufficient breadth to support the tongue without it slumping posteriorly, over either of the lateral edges of the blade.

With reference to FIGS. 6A, 6B, 9 and 10 of the drawings, the blade 5 in this embodiment comprises a pair of resiliently flexible elongate members 20, which may be formed from metal or relatively stiff plastic, that are secured at proximal ends thereof within fixed segment 7D and that extend, in the manner of spines, along respective sides of the blade 5, through segments 7C and 7B, and into segment 7A. Each of the segments 7A, 7B and 7C is fixed to the members 20. To enhance engagement between the segments 7A to 7D and members 20, the latter may be formed with a non-smooth exterior configuration, e.g. a serrated profile, along their lengths which forms axial interlock with the segments when moulded over the members 20.

The joints 8A, 8B, 8C, which are defined between the adjacent segments and which each have a length L_{J} of approximately half a centimetre, comprise sections 21 of the elongate members 20 which extend between the segments and thereby define ligaments. Because the sections 21 are resiliently flexible, they may bend to permit articulation of the segments when the curvature control mechanism is operated, as will be described in further detail later. The elongate members 20 will be of sufficient diameter, stiffness and hardness so that the sections 21 will not plastically deform during articulation and will exert a bias on the segments which is sufficient to restore the straight blade configuration when bending loads applied by the curvature adjustment mechanism are removed. The sections of casing 9 at the joints 8A, 8B, 8C are sufficiently thin and flexible to permit the articulation.

The laryngoscope 1 of this embodiment is a fibre-optic laryngoscope. To this end, the blade 1, with reference to FIG. 10 in particular, houses a fibre-optic wire 30 which, consistent with corresponding fibre-optic wires in traditional laryngoscopes, extends along the left-hand side of the blade to the blade tip, though in other embodiments may extend, for example, along the right-hand side of the blade or along the central longitudinal axis of the blade. The wire 30 extends through each of the four segments and through the handle for connection to a processing unit and video screen, which may be provided on the handle or provided separately from the laryngoscope 1 (see FIG. 8). A passage 31 extends through each segment and receives the wire 30 therein. The distal end of the wire 30 is positioned adjacent the open end of the passage 31 at the blade tip, where it is fixed. The wire 30 is a typical one used for the purposes of imaging the environment in the region of the blade tip. There is some slack in the wire 30 between its ends, and the wire 30 is slidably received in the passage 31 in each segment 7B, 7C and 7D, and loosely within the handle, so that the wire 30 can move freely when the segments are articulated.

The handle 5 comprises a cavity 6 which houses the wire 30 and a pulley system of the blade curvature adjustment mechanism which will be described in further detail later.

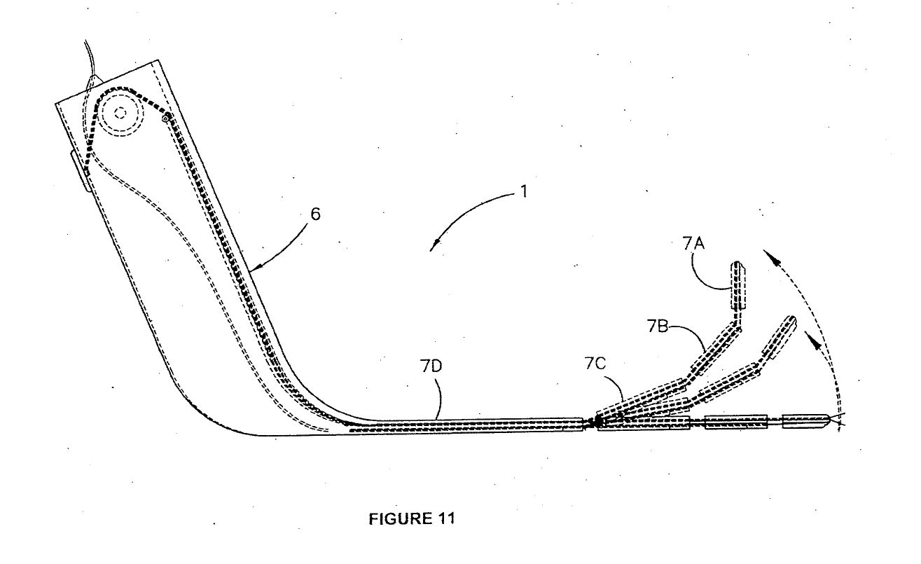

With reference in particular to FIGS. 11 and 13 to 16, the blade curvature adjustment mechanism 50 can be operated to adjust the curvature of the blade 5 during insertion of the blade into the buccal cavity/pharynx of a patient. Advantageously, in the present embodiment, the laryngoscopist can operate the mechanism 50 with the hand he or she uses to grasp the handle H. The curvature adjustment mechanism comprises an arrangement of tendons operable to effect angular displacement of the segments 7A, 7B and 7C about the joints 8A, 8B and 8C. Specifically, the mechanism comprises distal, middle and proximal tendons D, M and P respectively, which are anchored to segments 7A, 7B and 7C respectively at positions 56A, 56B and 56C respectively, those positions being anterior of/above the joints 8A, 8B and 8C and, more particularly, anterior of/above the hinges of the joints, which in this embodiment are living hinges defined by the resilient members 20. Distal tendon D extends from position 56A, which is near the tip of segment 7A (that segment having a length L_{SA} of about 1.5 cm), rearwardly along the length of the blade adjacent the anterior surfaces of the segments. Middle tendon M extends rearwardly from position 56B, which is approximately 4 mm rearward of the distal end of segment 7B (that segment having a length L_{SB} of about 2 cm), adjacent the anterior surfaces of the segments 7B, 7C and 7D. Proximal tendon P extends rearwardly from position 56C, which is approximately 14 mm rearward of the distal end of segment 7C (that segment having a length L_{SC} of about 3 cm), along the blade adjacent the anterior surfaces of segments 7C and 7D (the latter having a length L_{SD} of about 8 cm). Tendon M, proximal of its distal end overlies tendon D. In turn, tendon P, proximal of its distal end, overlies tendon M. The distal end of tendon D is fixed to the segment 7A at a position lying on a central plane passing through the blade orthogonal thereto. As shown in FIGS. 6A and 6B, the distal end of tendon M is bifurcated, so that it can be fixed to the anterior surface of segment 7B, at two points, unobstructed by tendon D, those two points being laterally equidistant from the central plane. Similarly, the distal end of tendon P is bifurcated, so that it can be fixed to the anterior surface of segment 7C, at two points, unobstructed by tendon M, those points also being laterally equidistant from the central plane. The tendons P, M, D extend in layered relation, along a line which lies in the central plane, to the cavity 6, axially along the cavity 6 adjacent a front wall thereof, over an idler roller 60, rotatably mounted in the cavity, and around a main roller 62, also rotatably mounted in the cavity, to an actuator 64 to which they are fixed at their distal ends. Tensioning of the tendons P, M, D results in even loading on each of the segments 7A, 7B and 7C about the central plane.

The blade 5 has a size corresponding to that of a size 4 Macintosh blade. In other embodiments, the blade may be smaller or larger, according to patient size or anatomy, but preferably, to this end, is scaled up or down, i.e. retains the same configuration and dimensional ratios disclosed herein. Of course, the passage of the sheath into which the blade is received, would then correspondingly be scaled up or down.

The actuator 64 in this embodiment comprises a thumb-operable slider which is retained by and axially movable along a track in a rear wall of the handle 6. As will be clear from FIG. 11, downward displacement of the slider 64, towards the blade, draws the tendons P, M, D around the rollers 60 and around the convex lower end of the cavity inner wall (which is smooth), thereby shortening the tendon length in the blade and effecting compression along an anterior side of the blade and thus bending of the joints and articulation of the segments. The mechanism is provided with a locking feature (not shown) by means of which the actuator 64 can be locked relative to the handle 6 at any position along the track, whereby straightening of the blade 5 is prevented. The locking feature may be one which can be disengaged by depressing the actuator 64 against a spring bias, thereby permitting sliding of the slider button, and engaged by simply allowing the slider button to revert to an undepressed position under the action of the spring bias.

In the present embodiment, the curvature adjustment mechanism is unpowered. However, in other embodiments, it may be powered.

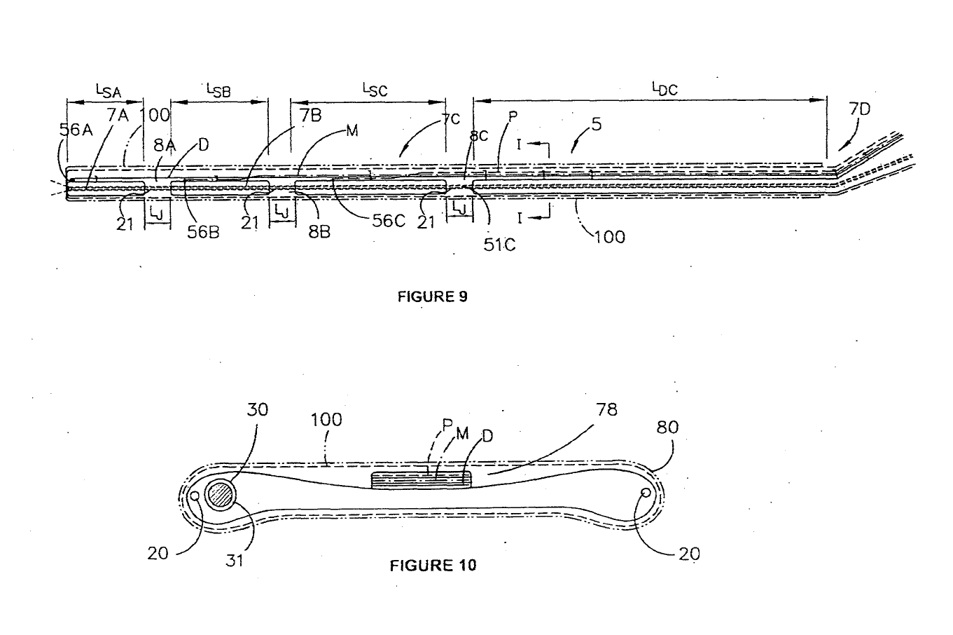

Referring to FIGS. 9 and 10, the tendons P, M, D are received through retainers arranged at spaced positions along the length of the blade. Tendon D is received through retainer 70 located on the anterior surface of segment 7A approximately halfway therealong, the retainer defining a bounded opening through which the tendon D is received, and providing a clearance in the direction normal to the plane of the segment 7A, whereby the tendon D can displaced anteriorly when tensioned to facilitate shortening of the length of tendon between position 56A and the joint 8A, without segment 7A and tendon D separating excessively. Retainer 72 is positioned on the anterior side of segment 7B, approximately halfway therealong, and defines three bounded openings 72A, 72B and 72C. The separate straight portions of the bifurcated end of middle tendon M are received through openings 72A and 72C, whilst the distal tendon D is received through opening 72B. Openings 72A and 72C have the same functionality in respect of tendon M as the opening 70A has in respect of tendon D, i.e. they allow limited anterior displacement of the tendon M (specifically its bifurcated end) when that tendon is tensioned, facilitating the requisite tendon length shortening between position 56B and joint 8B and thus angular displacement of segment 7B. Retainer 74 is disposed on the anterior side of segment 7C, approximately halfway therealong, and defines four bounded openings 74A, 74B, 74C and 74D. Openings 74A and 74D receive the straight portions of the bifurcated end of tendon P, permitting the limited anterior displacement of the tendon P from the segment 7C distal of anchor position 56C, and thus facilitating the necessary tendon length shortening between that position and the joint 8C for angular displacement of segment 7C. Tendons M and D extend through openings 74B and 74C respectively. Retainers 76 are disposed at spaced positions along the fixed segment 7D, each retainer 76 defining bounded openings 76A, 76B and 76C, through which tendons P, M and D respectively extend. The retainers 70, 72, 74 and 76, in addition to retaining the tendons individually, may also guide the tendons axially.

Owing to the positions at which the tendons are anchored to the segments, the distal segment 7A will be the segment which is angularly displaced, relative to the adjacent segment proximal to it (segment 7B in this case), at the greatest rate. This is advantageous because, during insertion of the laryngoscope blade, fine adjustment of the position and attitude of the leading end of the blade, such that it displaces the tongue and points directly towards the larynx, particularly in the latter stages of insertion, without giving rise to a sharp change in blade angle between the blade distal end and the remainder of the blade, is critical for ease of inserting the blade tip into the larynx. During insertion, the tongue will be cradled by the segments 7A, 7B, 7C and 7D, and will remain cradled as the blade curvature is adjusted. As the blade distal end draws near the larynx during insertion, and the position/angle of the distal segment is adjusted to be directed towards the larynx, the segments 7B, 7C and 7D, together with segment 7A, assume the profile of a curve, which, to all intents and purposes, remains substantially smooth, so that the laryngoscope blade can simply be advanced along the trajectory defined by the curve, into the larynx.

Referring to FIG. 8, retainers 76, of the type disposed on segment 7D, are arranged at spaced positions along the interior surface of the front wall of the handle 5 in the cavity 6, thus being inverted relative to the retainers on the segments, and retain, and possibly also guide, the tendons adjacent the front wall in exactly the same manner as the retainers 76 on the segment 7D retain/guide the tendons adjacent the anterior surface of that segment, albeit that the tendons are received through the openings in the retainers 76 in the cavity 6 in reverse order.

The distal end of the blade is provided with a light, which is located on or adjacent the distal end of the optic fibre camera/cable and provides illumination during insertion of the blade, facilitating appropriate adjustment of the blade curvature, especially the angle and position of the distal segment, which is carried out with the aid of the optic fibre camera, so that it points towards the larynx. Power is delivered to the light by a wire which preferably forms part of/is defined by cabling which comprises the fibre-optic camera wire, though may be a separate wire. The laryngoscope 1 is preferably provided with a battery (not shown) which powers the camera and light and which is preferably housed in the interior cavity in the handle. The battery may be rechargeable and, to this end, the laryngoscope may have a socket or other connector, preferably through a wall of the handle, which releasably receives a connector of a charging cable for recharging the battery.

A concavity 78 is formed along the anterior side of the blade (see FIG. 10), and the retainers, 70, 72, 74 and 76, which are provided on the segments are anchored at the bases of the concavities so as to be at least partially recessed or countersunk, whereby the overall depth of the blade can be kept to a minimum and the anterior surface of the blade as overlying any given segment is substantially flat, that surface being defined by a resilient skin or shell 80, formed by casing 9, which is received over the segments 7A to 7D and extends the length of the blade 5. As will be clear from FIG. 10, the edge portions of the segments, remote from the retainers, are relatively deep/thick so as to afford the blade adequate stiffness and strength.

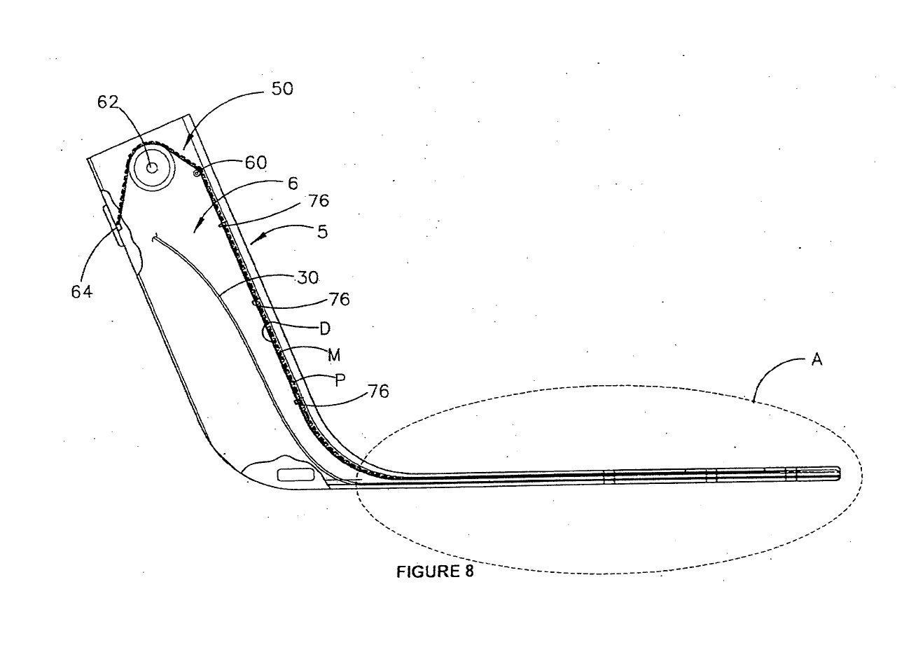

The laryngoscope 1 further comprises sheath 100, as briefly discussed previously, the sheath 100 being resiliently flexible and receivable over the blade 5 (see FIGS. 4, 5, 7 and 12). The sheath 100 may be formed from any suitable resiliently flexible material, particularly a plastics or rubber material, such as latex. The sheath 100 is formed with an anterior longitudinal passage 102, a posterior longitudinal passage 104 and a partition wall 106 separating the passages 102 and 104. Passages 102 and 104 are open at proximal ends thereof. Passage 102 is closed at its distal end by a wall 103, which is transparent to permit operation of the fibre optic camera and light when the blade is received in passage 102, as will be described in further detail shortly. Passage 104 is open at its distal end to permit intubation, as will also be described in further detail shortly.

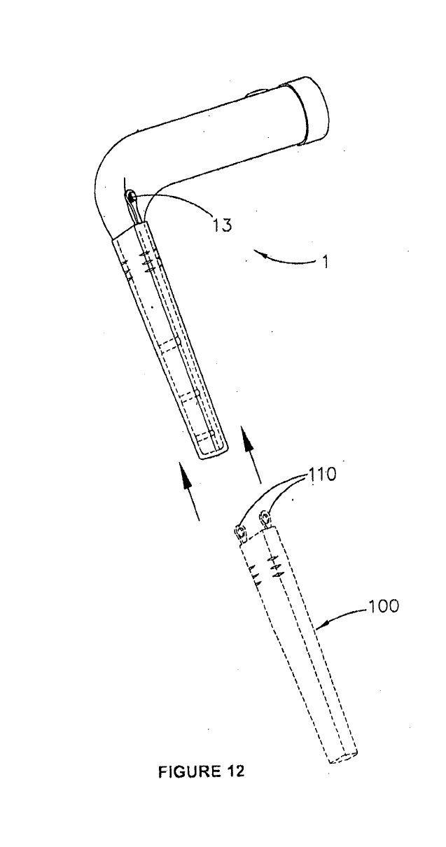

Referring to FIG. 12, prior to laryngoscopy being carried out, the blade 5, preferably in its straight configuration, is inserted into the passage 102 through its open distal end such that the passage 104 is positioned posterior to/beneath the posterior side (underside) of the blade 5. Elastic loops 110 formed integrally with the remainder of the sheath 100 and arranged at a proximal end of the sheath 100, to either side of the passages 102 and 104, are drawn rearwardly after insertion of the blade into passage 102, to receive the lugs 13 therethrough, thereby securing the sheath 100 to the blade 5. The sheath 100 remains over the blade 5 during insertion of the laryngoscope. Advantageously, because the sheath 100 covers the blade 5 completely, the blade 5, unlike that of a conventional laryngoscope, need not be sterilised. Instead, the sheath 100, which is designed for single use and is disposed of after the procedure, is provided sterilised, and will generally be provided as part of a plurality of pre-packed sterilised sheaths 100, in a similar manner to prepacked sterile latex surgical gloves.

The sheath 100 is sized and shaped so that it will stretch slightly to fit snugly both around and along the blade 5, thereby forming a thin “skin” which moves as one with the blade 5 during insertion and adjustment of the curvature of the blade.

Shallow notches 112 are formed in the outer wall of the sheath 100 near the proximal end of the sheath 100, where they are positioned to receive string for anchoring purposes, as will be described in further detail shortly.

Laryngoscopy is carried out using the laryngoscope 1 as follows.

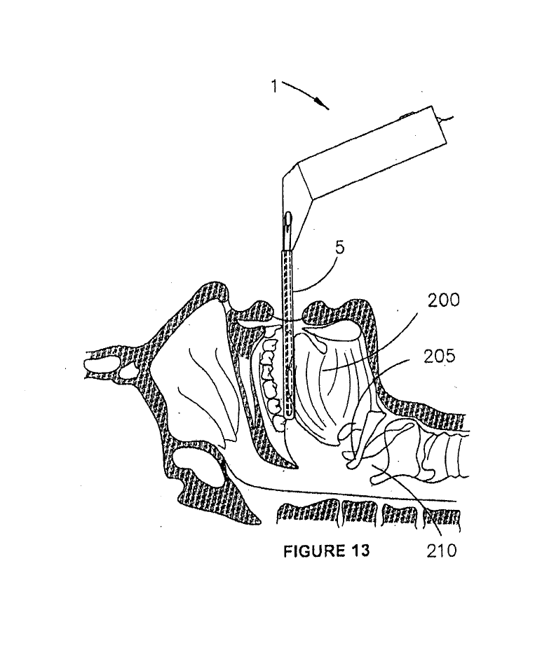

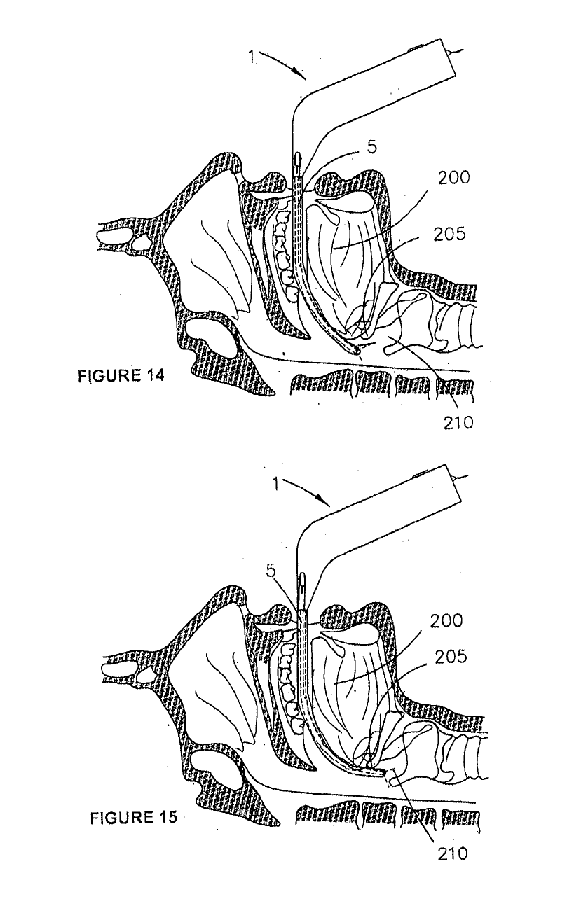

With the sheath 100 fitted to the blade 5 and secured to the laryngoscope 1 in the manner previously described and illustrated, the laryngoscopist grasps the handle, with the hand with which he/she is more dextrous, and inserts the blade into the patient's mouth (see FIG. 13), whilst holding the mouth open with his/her other hand. Advantageously, the blade can be centred over the midline of the patient's body throughout the procedure, owing to its symmetry. The laryngoscopist, using the thumb of the hand grasping the handle 6, moves the slider 64 towards the base of the handle 6, forcing the anterior face of the blade 5 towards the patient's torso and thereby lifting the patient's tongue 200 and mandible with a distal part of the blade 5 (see FIG. 14). Whilst applying continued pressure against the tongue 200 through the blade 5, towards a general centre of curvature of the curved portion of the blade, the laryngoscopist continues to insert the blade 5, with the aid of the light source and fibre optic wire (the operation of which is unhindered as a result of the end wall 103 of passage 102 being transparent). As the blade tip draws near the epiglottis 205, the laryngoscopist, by means of the camera/light source, can see whether the blade tip is pointing towards the trachea 210. In the exemplary scenario depicted in FIG. 14, it can be seen that the blade tip is pointed down too far so that, were the curvature of the blade 5 not adjustable, the necessary re-orientation of the laryngoscope 1 to point the blade tip in the correct direction may result in fouling of the laryngoscope 1 against the teeth/upper part of the mouth of the patient and/or flexion of the lower cervical vertebrae of the neck and extension of the head at the atlanto-occipital joint, the latter in particular being highly undesirable, particularly in the event of injuries to the neck/spine.

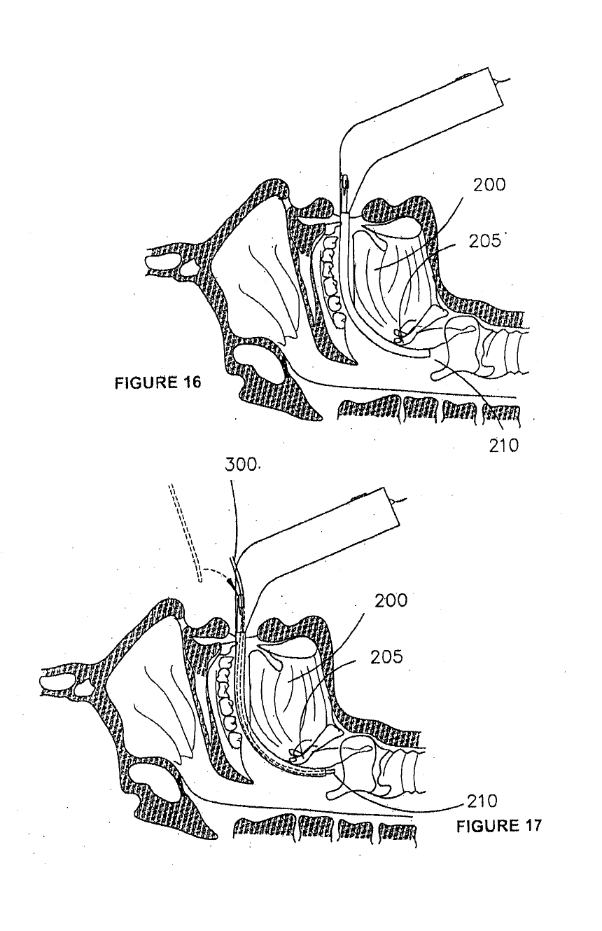

Referring to FIG. 15, the laryngoscopist, whilst maintaining the handle 6 and proximal portion of the blade 5 substantially where they are, depresses the slider 64 and draws it further towards the handle base, so that the blade tip points directly towards the trachea 210, the blade 5 thus lifting the epiglottis 205, whereupon the light source/optic fibre wire provide the laryngoscopist with a clear view of the trachea 210. Maintaining the requisite pressure against the tongue 200, the laryngoscopist then, along the trajectory defined by the blade curvature, inserts the blade 5 further so that its distal end enters the trachea 210 (see FIG. 16).

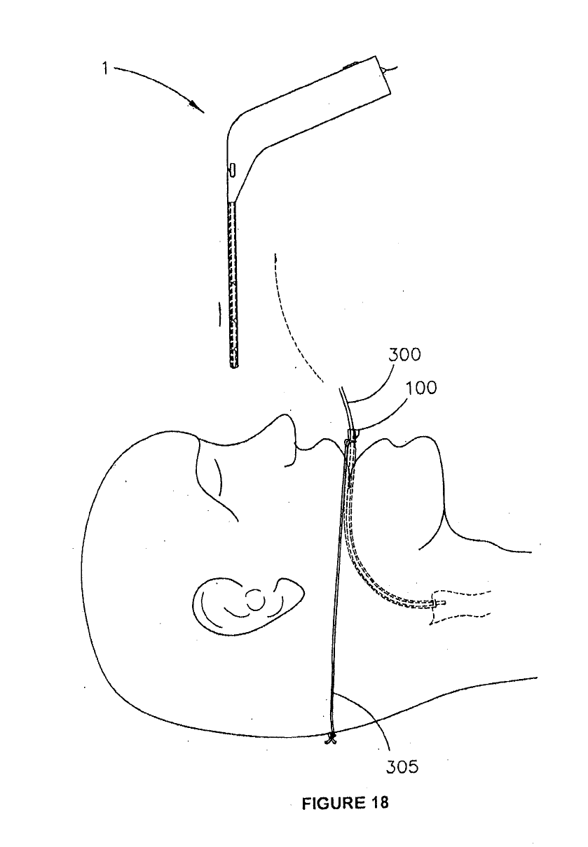

Referring to FIG. 17, having thus completed insertion of the blade 5, the laryngoscopist inserts a ventilation tube 300 into the proximal end of passage 104 and feeds it along that passage until its end projects through the distal end of the passage 104, thereby intubating the patient. Next, referring to FIG. 18, the laryngoscopist disengages the loops 110 from the lugs 13 and withdraws the blade 5 from the sheath 100, the latter remaining in position together with the tube 300, then wraps string around the sheath 100 in a manner such that it is received in notches 112, and tightens the string so that the sheath is scrunched against the tube 300, thereby being anchored to the tube by the string. The laryngoscopist then ties the string around the patient's head to anchor the sheath 100 and tube 300 in position.

In the following description of the second embodiment of the invention, the same reference numerals as have been used in relation to the first embodiment will be used to refer to corresponding features, but will be supplemented by an apostrophe.

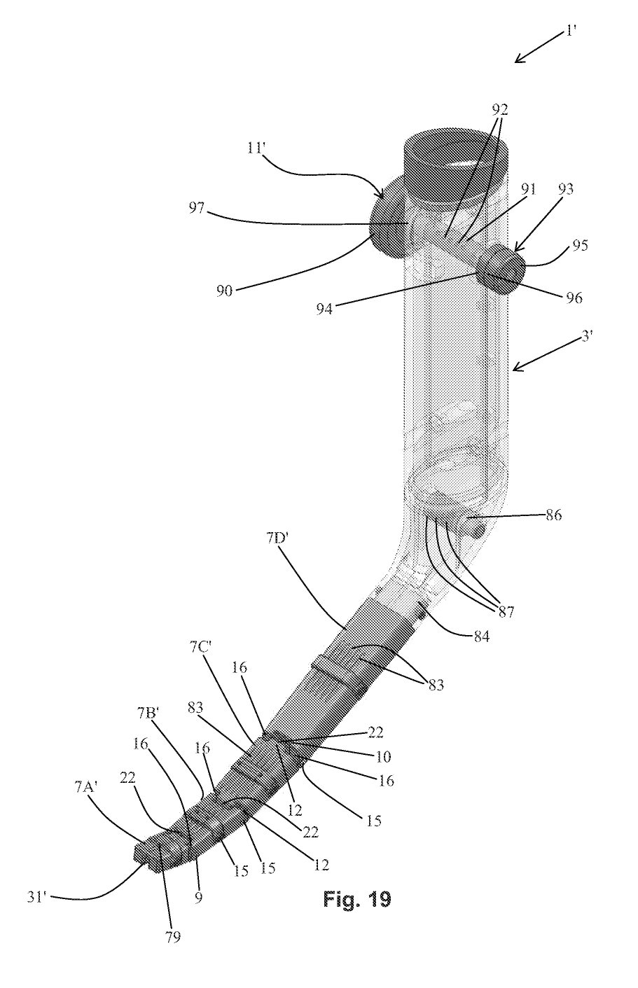

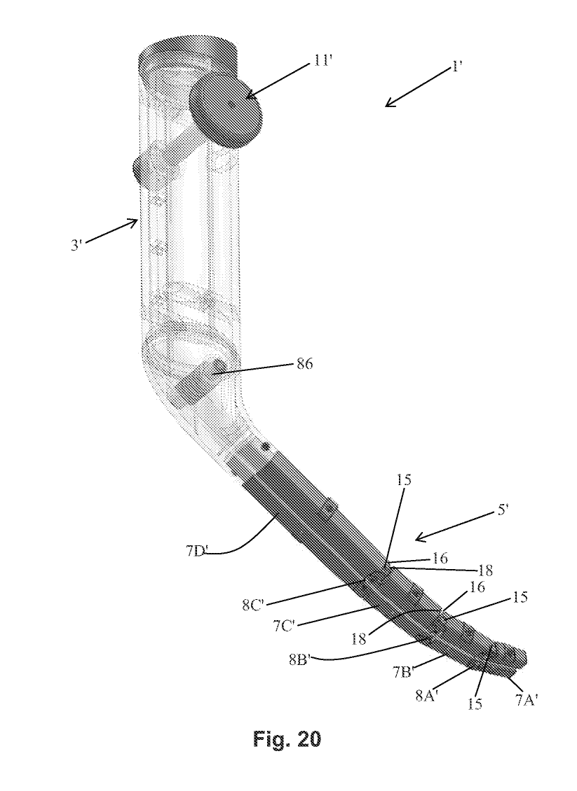

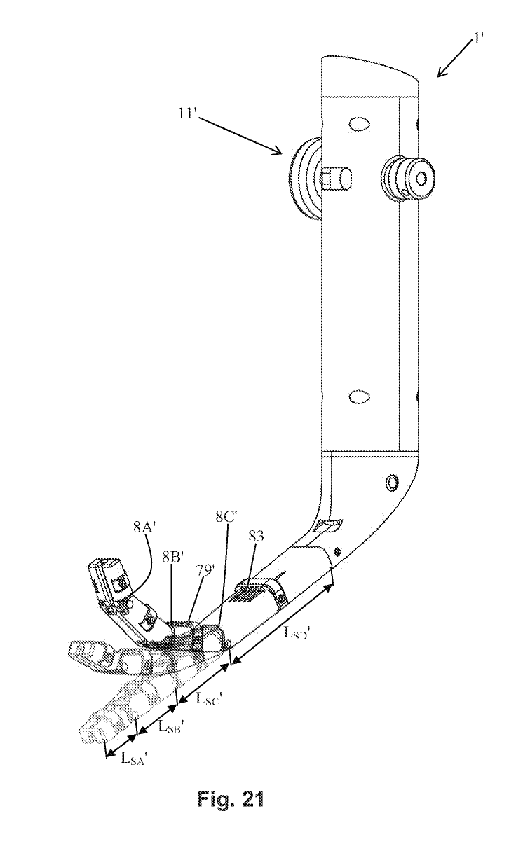

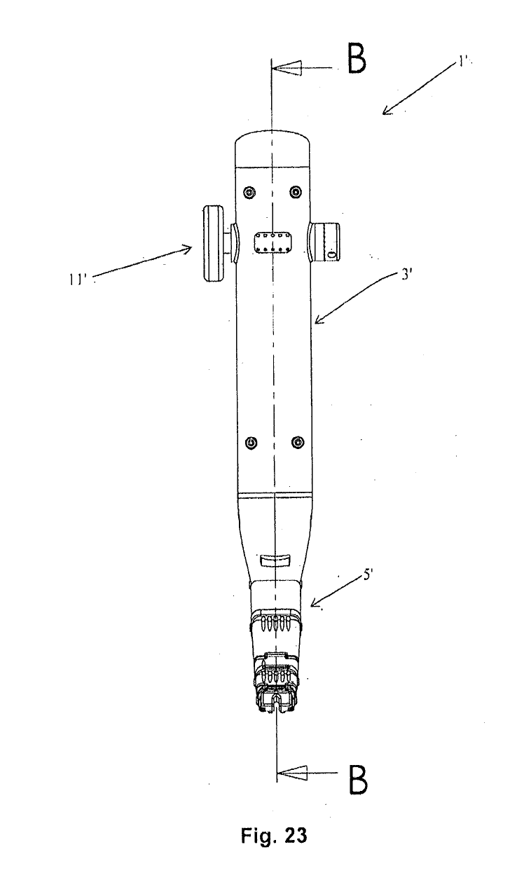

Details of a laryngoscope 1′ according to the second preferred embodiment are shown in FIGS. 19 to 25. The laryngoscope 1′, consistent with that of the first embodiment, includes a handle 3′ (shown transparent in FIGS. 19, 20 and 22 for clarity), blade 5′, only the segment/retainer assembly of which is shown in FIGS. 19 to 24, and actuator 11′ for adjusting the blade curvature.

The blade 5′, consistent with that of the first embodiment, comprises a plurality of segments 7A′, 7B′, 7C′ and 7D′ and interconnecting joints 8A′, 8B′ and 8C′. Segments 7A′, 7B′ and 7C′ are configured to pivot/articulate through operation of the actuator 11′.

In the present embodiment, the adjacent ends of adjacent segments receive respective pins 15 therethrough defining hinges about which the segments 7A′, 7B′ and 7C′ pivot/articulate. The segment 7B′ is configured at each end with a clevis 10, the distal one of which receives a boss 12 formed at the proximal end of segment 7A′, and the proximal one of which receives a boss 12 formed at the distal end of segment 7C′. The distal end of segment 7B′ is similarly formed with a clevis 10 which receives a boss 12 formed at the proximal end of segment 7C′. The pins 15 are received adjacent the posterior side of the segments and interconnected ends of the segments are configured such that there are clearances 14 therebetween anterior of the pins 15, which permit articulation of the segments. Each arm of each clevis 12 is bevelled so as to be provided with a sloped face 16 which, when the blade is straight, is spaced from a respective face 18 which is formed on the interconnecting segment laterally outward of the boss on that segment which the clevis receives, the face 18 being substantially orthogonal/square to the major plane of the respective segment on which it is formed. Correspondingly, each boss 12 is configured with a sloped face 22 which, when the blade is straight, is spaced from a respective face 24 on the adjacent interconnecting segment between the arms of the clevis 12 which receives that boss, each face 24 being approximately orthogonal/square to the main plane of the segment on which it is formed. The faces 16 and 18 are arranged so as to abut when the blade 5′ assumes its maximum curvature, as are the faces 22 and 24, whereby further increasing of curvature is precluded.

In this embodiment, the distance L_{SA}′ between the tip of segment 7A′ and the centre of pin 15 engaging it is about 1.6 cm, the distance L_{SB}′ between that pin centre and the centre of the pin 15 at the proximal end of segment 7B′ is about 2.3 cm, the distance L_{SC}′ between the latter pin centre and the centre of the final pin 15 is about 3.0 cm and the distance L_{SD}′ between that final pin centre and the proximal end of segment 7D′ is about 6.0 cm. These lengths may, alternatively, be the same, or nearly the same, value as L_{SA}, L_{SB}, L_{SC} and L_{SC} in the first embodiment.

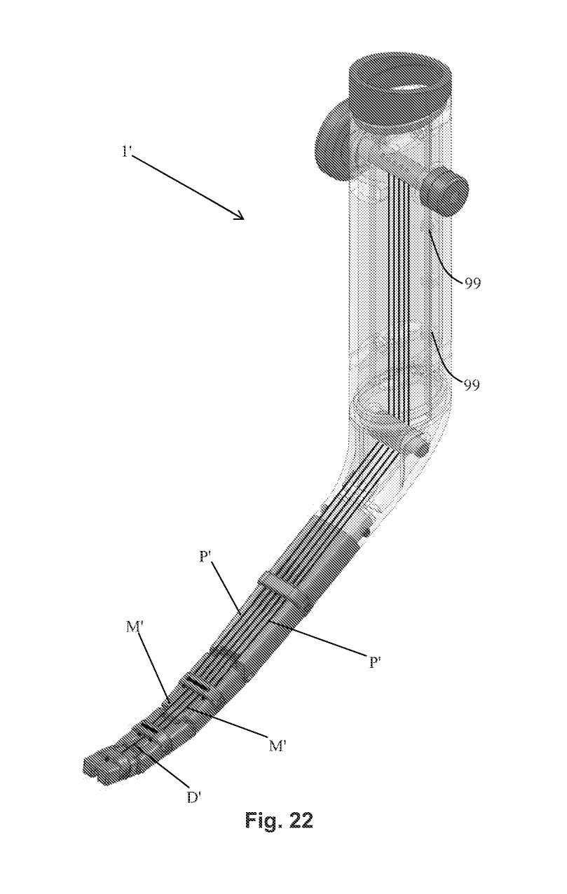

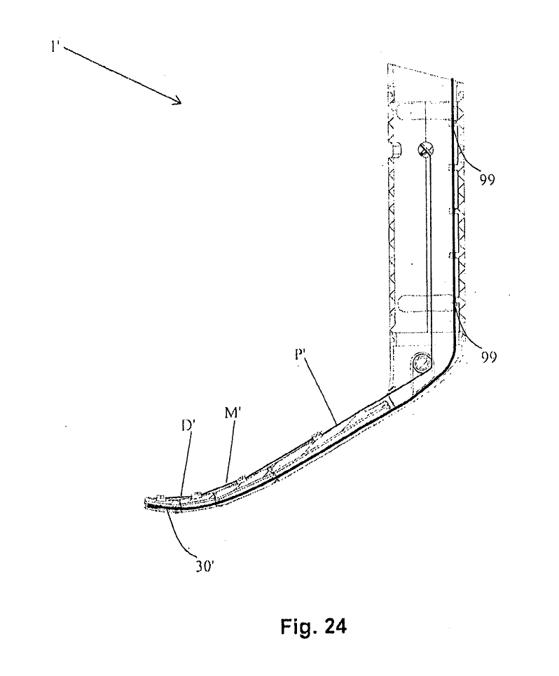

With reference to FIGS. 22 and 24, the curvature adjustment mechanism in the present embodiment, like that of the previous embodiment, includes a plurality of tendons which extend along the anterior face of the segments and are arranged to be drawn by the actuator 11′ to effect curvature of the blade 5′. The tendons in this embodiment are cables, which may be steel or titanium cables, and comprise a distal tendon D′ which is anchored to segment 7A′ a pair of middle tendons M′ which are anchored to segment 7B′ and a pair of proximal tendons P′ which are anchored to segment 7C. Distal tendon D′ extends rearwardly from along the length of the blade adjacent the anterior surfaces of the segments and extends along a central longitudinal axis of the blade. Middle tendons M′ are anchored to the segment 7B′ and are disposed to either side of the central longitudinal axis and spaced equidistant therefrom. The tendons M′ extend rearwardly adjacent the anterior surfaces of the segments 7B′, 7C′ and 7D′. The proximal tendons P′ are anchored to the segment 7C′ and are disposed to either side of the blade central longitudinal axis/central plane passing through the blade orthogonal thereto. Laterally outward of tendons M′, and spaced equidistant therefrom, are proximal tendons P′ which extend rearwardly along the blade adjacent the anterior surfaces of segments 7C′ and 7D′. In this embodiment, the tendons D′, M′, P′ are arranged in side-by-side relation.

The segments 7A′, 7W, 7C′ and 7D′ are provided with retainers 70′, 72′, 74′ and 76′ respectively, each retainer being configured in the form of a U-shaped member opposed arms of which are secured to opposite actual sides of the respective segment via threaded fasteners 77 such as screws. The lateral sides of the segments are formed with recesses, into which the legs are received, such that the legs are at least partially (though preferably fully) countersunk in the segments. Each retainer further comprises a cross member which extends between the legs and across the anterior side of the respective segment, each cross member being at least partially (though preferably fully) countersunk in a respective recess formed in the anterior side of the segment to which it is fixed. The cross member of retainer 70′ is formed with a downwardly opening channel through which distal tendon D extends and at a distal end of which the tendon D′ is secured; the retainer 70′ is provided solely for the purposes of anchoring the tendon D′ to the segment 7A′. Retainer 72′ is formed with three such channels 79, a central one of which receives distal tendon D′ slidably therethrough and laterally outer ones of which receive middle tendons M′. The cross member of retainer 72′ is formed with a pair of holes 81 therethrough. The tendons M′ are defined by sections of a single length of cable which is trained through the holes 81 and over the outer surface of the cross member such that distal ends of the tendons M′ are anchored to the segment 7B. Retainer 76′ is formed with five such channels 79, the central one of which receives tendon D′ slidably therethrough. The channels 79 adjacent the central one on either lateral side thereof receive tendons M′ slidably therethrough. The laterally outermost channels receive tendons P′ therethrough. Tendon P′, like tendon M′, is formed from a single length of cable which is similarly trained through holes 81 in the cross member of a retainer 76′ whereby the distal ends of the tendons P′ are anchored to the segment 7C. The cross member of retainer 76′ is also formed with five channels 79. The tendon D′ is slidably received through the central channel 79, the tendons P′ are slidably received through the laterally outermost channels 79 and the tendons M′ are slidably received through the other two channels 79. Each of channels and the segment to which it is fixed define bounded openings through which a tendon is received, providing a clearance in the direction normal to the plane of the respective segment, whereby the tendon can be displaced anteriorly when tensioned to facilitate curvature adjustment, and particularly the shortening of the lengths of tendon necessary for the blade curvature to be increased.

Longitudinal channels or grooves 83 are formed in the anterior surfaces of each segment and at least partially receive sections of the respective tendons which extend over the anterior surface of the respective segment and through the respective channels 79, permitting the degree to which each retainer cross member sits proud of the anterior surface to be minimised. The sections of tendon slidably received through the retainer channels 79 can be displaced transverse to the longitudinal blade axis owing to there being clearances at the positions where they are retained in channels 79/grooves 83.

The tendons extend through an opening 85 at a front lower end of the handle and are trained around a sheave 86 rotatably supported within the handle, the sheave 86 being formed with a plurality of circumferential grooves 87 each of which receives a respective tendon. The tendons extend from sheave 86 to the actuator 11′ which, in this embodiment, comprises a thumb-operable wheel 90, a spool defined by a shaft 91 to which the wheel is fixed and which extends through the handle 3′, via which the wheel is rotatably mounted to the handle 3′. Holes 92 are formed through the shaft 91 and fixedly receive upper ends of the tendons. The actuator 11′ further comprises a ratchet mechanism 93 arranged on the opposite lateral side of the handle to the wheel 90, the ratchet mechanism comprising a first cylindrical element 94 which is fixed with respect to the handle 3′ and a second cylindrical element 95 which is fixed to an end of the shaft 91, the elements 94 and 95 being formed with interengaging serrations or teeth 96 which, whilst engaged, permit the wheel 90 to be rotated in the direction which tensions the tendons (and thus effects curvature of the blade) but not in the other direction. The user of the laryngoscope, while grasping handle 3′, can engage a rear portion of the wheel 90 with their thumb and draw it in a downward/posterior direction to effect partial winding of the tendons around the shaft 91 and thus curving of the blade 5′.

The shaft 91 can float axially to a degree within the handle 3′ such that depression of the wheel 90, such as by finger or thumb pressure applied to the laterally outer face thereof, will displace the shaft 91 and thus also element 95, such that the latter is disengaged from the element 94 and the shaft 91 and wheel 90 may rotate to permit unwinding of the tendons from the shaft 91 and thus resumption of the straight blade configuration. A helical spring 97 is interposed between the wheel 90 and handle 3′, the spring being received around shaft 91 and exerting an outward bias against wheel 90 to hold the elements 93 and 94 in engagement, which bias is overcome by the aforementioned depression of the wheel 90.

In this embodiment, there is no biasing means which acts to bias the blade towards a relaxed/straight configuration, though the embodiment may be modified such that there is such a biasing means, without departure from the invention.

The segments, retainers and screws which hold the retainers to the segments are preferably titanium. The handle may be plastic, and is formed at its lower end with a recess 51 into which a spigot 84 which is integrally formed with and projects rearwardly from the segment 7D′. Screws received through lateral side walls of the handle casing at the lower end engage with threaded holes in the lateral sides of the spigot 84 to secure the spigot in the recess and thus the blade to the handle.

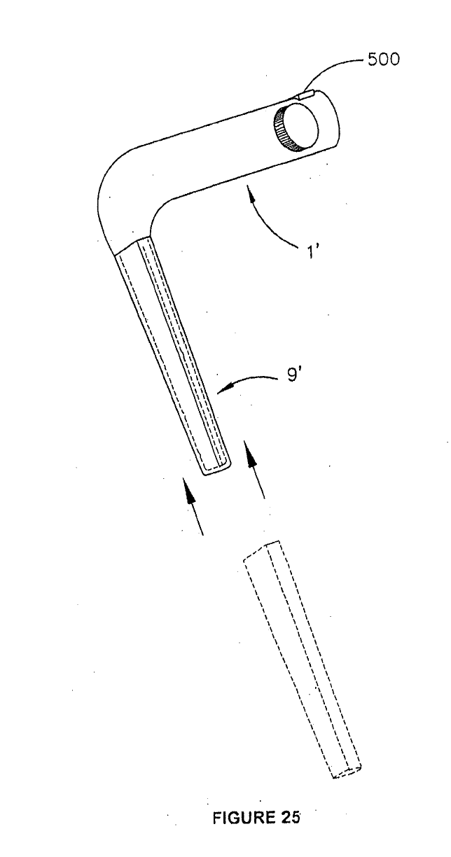

A posteriorly opening channel 31′ formed into the undersides of the segments extends the length of the blade 5′, along a central longitudinal axis of the blade and receives the wire of a fibre optic camera 30′, which is shown in FIG. 24. The wire, consistent with that in the previous embodiment, extends into the handle for connection to a processing unit (not shown) and video screen 500, the latter being shown in FIG. 25, which form part of the laryngoscope 1′, whereby an image of the region into which the blade is being inserted/towards which the blade is directed can be displayed on the screen. The screen and processing means may alternatively be provided separately from the laryngoscope and the laryngoscope adapted for connection thereto to couple operatively the camera and processing means/screen. The distal end of the wire is positioned adjacent the open end of the passage 31′ at the blade tip, where it is fixed. Again, the imaging wire is a typical one used for the purposes of imaging the environment in the region of the blade tip, and there is some slack in the wire between its ends and the wire is slidably received in the passage 31′, and loosely within the handle, so that it can move freely when the segments are articulated. With reference to FIG. 24, the cabling for the light/camera 30 is held to the rear wall of the handle casing interior by a plurality of spaced apart resilient clamping members 99 which are integrally formed with that wall.

With reference to FIG. 25, the blade 5′ further comprises a resilient casing/skin/shell 9, which may be formed from plastic, closely received over the segment/retainer assembly to afford the blade a smooth exterior profile, the casing 9′ being sufficiently flexible to permit articulation of the segments. The casing 9′ may be removably received over the segment/retainer assembly as illustrated, or instead permanently received over that assembly. The casing is configured so as not to interfere the operation of the light/camera at the blade distal end. For this purpose, it may be configured with an opening at that end or cover the end but be transparent. In the latter case, it will have preferably have minimal/negligible thickness where it covers the end.

In an alternative embodiment, there is no such casing, and the blade is constituted solely by the segments. The blade in this embodiment is configured such that the retainers do not project proud of the exterior surfaces of the segments, whereby the blade is generally smooth. In either case, the overall configuration and dimensions of the blade may be the same as those of the blade in the first embodiment.

The blades of the laryngoscope 1′ and the laryngoscope described in the immediately preceding paragraph both likewise receive a sheath as previously described and illustrated (the handle, to this end, in each being provided with lugs as previously described/illustrated).

In the following description of the third embodiment of the invention, the same reference numerals as have been used in relation to the first embodiment or second embodiment will be used to refer to corresponding features, but will be supplemented by two apostrophes or an apostrophe respectively.

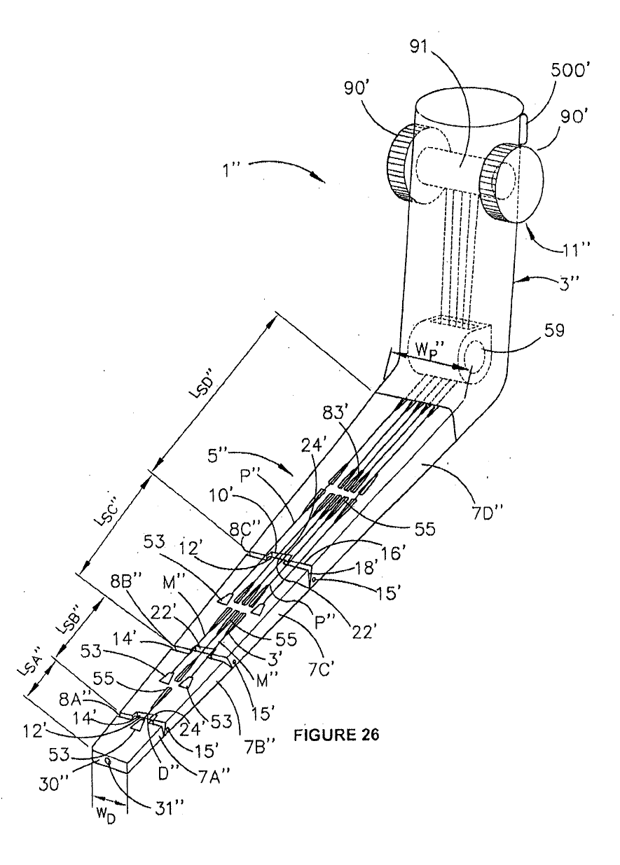

A laryngoscope 1″ according to a third preferred embodiment is illustrated in FIG. 26. The laryngoscope 1″ is similar to the laryngoscopes 1 and 1′, comprising an actuator 11″ which in this embodiment comprises a pair of wheels 90′—one either side of the handle 3′.

The laryngoscope 1″ further comprises a tensioner, through/around which the tendons D″, M″ and P″ are trained, for maintaining the lengths of the tendons which extend therefrom along the blade taut.

The actuator 11″ is preferably provided with an appropriate releasable ratchet mechanism (not shown) for maintaining the lengths of the tendons D″, M″ and P″ extending between the tensioner 59 and the shaft/spool 91.

The blade 5″ of the laryngoscope 1″ comprises segments 7A″, 7B″, 7C″ and 7D″, these segments defining the blade 5″ (there being no covering or casing thereover). The segments, like those of the laryngoscope 1′, are formed from metal which is preferably titanium. The blade 5″, like those of the other embodiments, may vary in size according to the size/anatomy of the patient. In the example illustrated, the lengths L_{SA}″, L_{SB}″, L_{SC}″ and L_{SD}″ are the same, or nearly the same, as the lengths L_{SA}, L_{SB}, L_{SC} and L_{SD} in the first embodiment, and the widths W_{D}″ and W_{P}″ are the same, or nearly the same, as the widths W_{D} and W_{P} respectively in the first embodiment. The blade 5″ is of progressively reducing thickness in the direction from its proximal end to its distal end, though alternatively may be of constant thickness consistent with the previous embodiments. Preferably, the maximum blade thickness does not exceed about half a centimetre.

Alternatively, the actuator may comprise a single wheel in driving engagement with the shaft/spool 91, most of which wheel is housed within the handle, a rear portion of the wheel protruding slightly outwardly from the handle in a rearward direction to be thumb-engageable, the wheel being disposed on/over a central longitudinal axis of the handle.

The segments, consistent with those in the laryngoscope 1′, are formed with bosses/clevises and the associated abuttable faces 16′, 18′ and 22′, 24′ to preclude overcurving of the blade.

In this embodiment, the blade 5″ is not provided with separate retainers. Instead, the distal ends of the tendons are anchored, by any suitable means, to the segments at anchor points 53, and the segments are formed with longitudinal passages 55 proximal and distal ends of which open out onto the segment/blade anterior surface, through which the tendons are trained, whereby the tendons are slidably retained within anterior portions of the segments, those portions defining retainers. Each segment is preferably formed with grooves 83′ which function in a manner analogous to that of grooves 83 in the laryngoscope 1′F.

Formed through/into the segments/blade is a longitudinal passage 31″, along which cabling for the light and camera extends, consistent with embodiments as previously described. The laryngoscope 1″ further includes video screen 500′ and an associated processing unit, which, like that in the previous embodiment, is preferably housed within the handle 3″.

The laryngoscope 1″ preferably includes an onboard power source, such as a battery, which is preferably rechargeable, to power the camera/light/screen.

The blade 5″, like that of the previous embodiments, receives a sheath as previously described and illustrated (the handle, to this end, being provided with lugs as previously described/illustrated). The arrangement of tendons/anchor points/joints on the blade does not have any parts which are to a significant extent proud of the blade anterior surface, nor any sharp projections or edges, whereby the sheath will not be damaged notwithstanding its being in contact with the assembly in use.

In each of the embodiments, the handle is preferably a “stubby handle”, preferably having a length of about 10 centimetres, whereby functionality of the laryngoscope during blade insertion is optimised. In those embodiments which incorporate a video screen, the screen may be located elsewhere on the handle if necessary, e.g. on the top of the handle (to face generally upwardly), to facilitate viewing thereof.

In an alternative embodiment, the blade may be of unitary/single-piece construction and resiliently flexible, so that the segments are integrally formed (and may thus be notional segments) and the joints are defined by portions of the blade (and thus may be notional joints) which resiliently flex/deform upon operation of the means for adjusting curvature to increase the curvature, the resilient flexibility of the blade, in such an embodiment, preferably then biasing the blade into a relaxed configuration, which is preferably a straight configuration.

It will be appreciated that a sheath/fitting of the type disclosed herein is, without departure from the invention, applicable not only to laryngoscopes embodying the invention but also to laryngoscopes generally (including ones the blade curvature of which is not adjustable).

The laryngoscope of each embodiment and sheath offer a number of advantages over conventional laryngoscopes, including video laryngoscopes such as Cemach, McGrath, Airtraq and Glidescope laryngoscopes. The minimal thickness of the blade allows easy insertion into the airway, with minimal mouth opening being necessary and dental trauma being avoidable. Moreover, neck movement is avoidable because forces can be applied along the length of the blade, under direct vision, to the tongue and anterior structures. Also, cardiovascular responses to intubation can be reduced, whereby the procedure may be safer in patients with heart disease or raised intracranial pressure, and may possibly be carried out on an awake or minimally sedated patient. Furthermore, the laryngoscope 1, which is configured for operation with either the left or right hand, provides the benefit of familiarity of technique. In addition, because the impermeable sheath 100 encloses the blade 5, the need for expensive sterilisation procedures is reduced or eliminated. Finally, intubating conditions are improved generally, owing to ease of blade insertion, increased blade manoeuvrability, an improved view during insertion, reduced nervous stimulation of the patient, and ease of intubation under direct vision.

Many modifications will be apparent to those skilled in the art without departing from the scope of the present invention. Features of one embodiment may be readily combined with features from one or more other embodiments, without departure from the invention. Each embodiment in this specification is to be applied mutatis mutandis to every other embodiment unless expressly stated otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A laryngoscope comprising:
a blade having an adjustable curvatures; and
a curvature adjustment device to adjust the curvature of the blade which is operable during insertion of the blade into the airway of a patient such that the blade lifts a tongue and anterior structures of the patient to facilitate the insertion.

2. A laryngoscope according to claim 1, wherein the curvature adjustment device comprises a mechanism operable to effect axial compression adjacent an anterior side of the blade to increase blade curvature.

3. A laryngoscope according to claim 1, wherein the blade comprises a proximal section which is adjacent a handle of the laryngoscope and fixed with respect thereto, and a distal section extending from the proximal section, the distal section being angularly displaceable relative to the proximal section via the curvature adjustment device whereby the blade curvature is altered.

4. A laryngoscope according to claim 1, wherein the blade includes a plurality of portions therealong which are displaceable by the curvature adjustment device at rates which increase progressively from a proximal one of the portions to a distal one of the portions

5. A laryngoscope according to claim 1, wherein the blade comprises a plurality of segments arranged in end-to-end relation in a direction from a proximal end of the blade to a distal end of the blade, and at least one joint, the or each joint interconnecting the segments in a respective pair of adjacent segments, a distal segment in the or each pair being engaged with the curvature adjustment device, whereby operation of the curvature adjustment device effects articulation of the distal segment(s) about the joint(s).

6. A laryngoscope according to claim 5, wherein the blade comprises four said segments and thus three said joints.

7. A laryngoscope according to claim 5, wherein the curvature adjustment device comprises a mechanism including at least one tendon which is anchored at a distal end thereof to the distal segment in a respective said pair and extending across, and anterior of, a respective articulation axis of the distal segment defined by the respective joint, and which is coupled at a proximal end thereof to an actuating device which is operable to effect tensioning of the tendon(s) and thus articulation of the distal segment(s) about the joint(s).

8. A laryngoscope according to claim 7, comprising a retaining device which retains the tendon(s) in close proximity to the segments along the length of the blade, the retaining device comprising at least one retainer received over the tendon(s), with respect to which the tendon/s is/are axially movable.

9. A laryngoscope according to claim 8, wherein the retaining device comprises at least one said retainer on the proximal segment in the or each said pair received over the tendon(s) anchored to the distal segment in that pair, with respect to which retainer(s) the tendon/s is/are slidably movable.

10. A laryngoscope according to claim 5, wherein the blade comprises a pliable or flexible covering over the segments.

11. A laryngoscope according to claim 7, wherein the blade comprises a pliable or flexible covering which extends over the segments and the tendon.

12. (canceled)

13. A laryngoscope according to claim 1, wherein the curvature adjustment device includes an actuator which is operable by a digit on the hand which holds a handle of the laryngoscope.

14. A laryngoscope according to claim 13, wherein the actuator is operable by the thumb on the hand which holds a handle of the laryngoscope.

15. A laryngoscope according to claim 1, further comprising a ratchet wherein the curvature adjustment device is configured to be locked after each operation thereof to retain blade curvature.

16. A laryngoscope according to claim 15, wherein the curvature adjustment device comprises a releasable ratchet configured to retain blade curvature after each operation of the curvature adjustment device.

17. A laryngoscope according to claim 1, wherein the blade is generally symmetrical and planar.

18. A laryngoscope according to claim 1, being configured for left-hand or right-hand operation.

19. A laryngoscope according to claim 1, further comprising a light source operable to provide illumination to facilitate insertion of the blade.

20. A laryngoscope according to claim 1, further comprising a camera operable to output a view of the airway to a screen.

21. A laryngoscope according to claim 20, incorporating said screen.

22. A laryngoscope according to claim 20, wherein the camera comprises an optical fiber extending along the blade.

23. A fitting for a laryngoscope which when in use is received by the laryngoscope blade to define a guide along which an endotracheal tube may be fed to intubate a patient following insertion of the laryngoscope into the patient's airway to provide a view of the patient's vocal cords, the fitting being releasable from the blade to permit withdrawal of the blade from the fitting and thus from the airway.

24. A fitting according to claim 23, the fitting comprising a passage in which the blade is received, the passage being open at a proximal end thereof to permit the blade to be inserted thereinto and withdrawn therefrom.

25. A fitting according to claim 24, wherein the passage is defined by a sleeve.

26. A fitting according to claim 23, being configured to form a liquid/pathogen-impermeable barrier over the blade.

27. A fitting according to claim 23, including a passage which, when the fitting is in use, extends in alignment with the blade and is open at opposite ends, the passage being arranged for insertion of the tube therethrough.

28. A fitting according to claim 27, wherein the passage which is open at opposite ends is arranged to be disposed on a posterior side of the blade when the fitting is received by the blade.

29. A fitting according to claim 23, being of sheath-like form.

30. A fitting according to claim 23, being formed from an elastomer.

31. A fitting according to claim 23, which is securable to the tube to prevent axial movement of the fitting relative to the tube following withdrawal of the blade from the fitting.

32. A combination comprising a fitting according to claim 23 and said laryngoscope.

33. A combination according to claim 32, wherein the laryngoscope comprises a blade having an adjustable curvature and a curvature adjustment device to adjust the curvature of the blade which is operable during insertion of the blade into the airway of a patient such that the blade lifts the tongue and anterior structures of the patient to facilitate the insertion.

34. A laryngoscopy procedure, comprising inserting the blade of a laryngoscope into the airway of a patient, and adjusting the curvature of the blade such that the blade lifts the tongue and anterior structures to facilitate the insertion.

35. A procedure according to claim 34, wherein the laryngoscope comprises a blade having an adjustable curvature and a curvature adjustment device to adjust the curvature of the blade which is operable during insertion of the blade into the airway of a patient such that the blade lifts the tongue and anterior structures of the patient to facilitate the insertion.

36. An endotracheal intubation procedure comprising:
effecting receipt, by a blade of a laryngoscope, of a fitting to define a guide along the blade;
inserting the blade into the airway of a patient to obtain a view of the patient's vocal cords;
feeding the tube along the guide to intubate the patient; and
withdrawing the blade from the airway in a manner which releases it from the fitting, whereby the fitting remains inserted in the airway with the tube.

37. A procedure according to claim 36, comprising securing the fitting to the tube after withdrawal of the blade to preclude relative axial movement between the fitting and tube.

38. A procedure according to claim 36, wherein insertion of the blade into the airway of the patient is effected via a procedure comprising adjusting a curvature of the blade such that the blade lifts a tongue and anterior structures of the patient to facilitate the insertion of the blade.

39. A procedure according to claim 36, wherein the fitting is releasable from the blade to permit withdrawal of the blade from the fitting and the airway.
